(19) United States
(12) Patent Application Publication
(10) Pub. No.: US 2005/0215890 A1
(43) Pub. Date: Sep. 29, 2005

(54) First pass and/or gated blood pool imaging system including multiple camera heads

(76) Inventors: Lon P. Wilson, Irwin, PA (US); Thomas P. Power, Wexford, PA (US); Angelo Skulas, Fort Lauderdale, FL (US)

Correspondence Address: Alan G. Towner, Esquire;Pietragallo, Bosick & Gordon, One Oxford Centre, 38th Floor, 301 Grant Street, Pittsburgh, PA, 15219, US

(21) Appl. No.: 10/983,381

(22) Filed: Nov. 8, 2004

Related U.S. Application Data
(60) Provisional application No. 60/518,587, filed on Nov. 7, 2003.

(51) Int. Cl. A61B 6/00

(52) U.S. Cl. 600/436

(57) ABSTRACT

A cardiac imaging system comprising two or more camera heads is disclosed. Each camera is capable of generating first pass and/or gated blood pool images. One of the camera heads may be oriented at an anterior position with respect to the patient, and the other camera head may be oriented at a side position.

### CROSS-REFERENCE TO RELATED APPLICATION

This application claims the benefit of U.S. Provisional Patent Application Ser. No. 60/518,587 filed Nov. 7, 2003, which is incorporated herein by reference.

This application is related to U.S. application Ser. No. ______ filed Nov. 8, 2004 entitled “Gated Blood Pool and First Pass Imaging System”, which claims the benefit of U.S. Provisional Application Ser. No. 60/518,203 filed Nov. 7, 2003, both of which are incorporated herein by reference.

### FIELD OF THE INVENTION

The present invention relates to cardiac imaging, and more particularly relates to a first pass and/or gated blood pool imaging system including multiple cameras.

### BACKGROUND INFORMATION

Nuclear cardiac imaging procedures include first pass, gated blood pool, and single photon emission computed tomography (SPECT) imaging. These procedures are described in detail in a book by Carol English et al. entitled Introduction to Nuclear Cardiology, Third Edition, 1993, which is incorporated herein by reference. Summaries of first pass, gated blood pool, and SPECT imaging procedures are described below.

### First Pass

In standard first pass studies, the anterior view is routinely performed because resolution and count rate are maximal with this view, it is easy to perform during exercise, it provides good separation between the heart and lungs, it is satisfactory for analysis of segmental wall motion, and it permits assessment of both right and left ventricular parameters with a single injection.

The camera scintillation used in first-pass studies must be capable of providing adequate temporal and spatial resolution with acceptable counting statistics. Conventional single-crystal cameras are limited in their count rates to about 60,000 counts/sec, after which dead-time losses and data distortion occur. Conventional multicrystal cameras permit higher count rates, e.g., 450,000 counts/sec or higher, without significant dead-time losses. Multicrystal systems may utilize an array, e.g., ranging from 294 to 400 individual sodium iodide crystals and may be coupled by means of a light pipe array, e.g., ranging from 35 to 115 photomultiplier tubes. Because event detection and positioning are independent, the dead time of the system is almost exclusively a function of the speed at which the electronics can process an event.

The acquired data are usually stored in list mode or frame mode at 20 to 25 frames/sec (40 to 50 msec intervals). This allows accurate analysis of the high-frequency components of the ventricular time-activity curves. The optimal framing rate is a function of heart rate. The higher the heart rate, the greater the framing rate must be to obtain the same temporal resolution. At higher heart rates (i.e., during exercise), a given framing rate results in fewer counts per frame, which may yield statistically invalid results. This problem may be resolved by using a higher-sensitivity collimator or by increasing the radionuclide dose.

Subtraction of background activity from overlying and underlying blood-containing structures is also important for accurate determination of left ventricular functional parameters. A time-activity curve may be used to aid in determining the background activity. The operator may select a series of frames just prior to the first visible left ventricular beat on the time-activity curve. This frame preference provides a constant background image that represents overlying and scattered radiation from the left atrium and lungs at which time the radionuclide is present in these structures. The result is a regional background correction that enhances the definition of left ventricular edges.

Once the data have been collected and reviewed, a computer is used to derive indices of left ventricular function. This requires that the operator first select a region of interest (ROI) over the left ventricle corresponding to the end-diastolic image. The activity in this region is analyzed only during the time when it is in the left ventricle. Careful definition of the left ventricular ROI is crucial since over-or under-estimation results in inaccurate determinations.

Once the region of interest has been defined and the background subtracted, the left ventricular ejection fraction is calculated as the difference between end-diastolic counts (EDC) and end-systolic counts (ESC), divided by the end-diastolic count. Counts can be calculated using either an average of several beats or the summed cardiac cycle produced by several beats added frame by frame. For statistical reasons, only the beats at the peak of the time-activity curve should be used for data analysis. In patients with premature ventricular contractions, the premature beat and the post-premature beat should be excluded from the analysis since they do not reflect accurate values of ejection when compared to normal conduction patterns. However, such abnormal beats can provide valuable stroke volume data when analyzed on an individual beat-to-beat basis and compared to normal beats. The statistical error in calculating the left ventricular ejection fraction is a function of errors in determining the EDC, ESC and background counts. The error may be lessened by taking an average of several beats or forming a summed representative cycle.

Other ejection-phase indices of left ventricular performance can also be calculated from the first-pass data. The shape of the ventricular volume curve can tell much about the rate of ventricular emptying and filling, which may indicate valvular or compliance abnormalities which can affect global left ventricular performance.

Using a summed representative cycle also permits the evaluation of regional wall motion by observing the summed images. For a more accurate assessment of wall motion, especially in patients with coronary artery disease, studies may be performed in more than one view. In conventional systems, this requires multiple injections of a tracer that is rapidly cleared from the blood, with sequential studies in several views, or use of a dual-angle bilateral collimator.

Using conventional angiographic area-length geometric approximations, the operator can determine end-diastolic volume (EDV) from the end-diastolic image of the summed cardiac cycle. And just as in angiography, it is relatively easy to determine the end-systolic volume (ESV), stroke volume, cardiac output and pulmonary blood volume.

First-pass studies can also yield valuable information about right ventricular function. In the anterior position there is some anatomic overlap of the right atrium onto the right ventricle, contributing right atrial background to the right ventricular time-activity curve. To correct this problem, a background region of interest may be selected adjacent to the right ventricle at the interface between the right ventricle and the right atrium. A high frequency time-activity curve is generated and temporally subtracted from the right ventricular time-activity curve. The right ventricular volume curve can then be used to determine the right ventricular ejection fraction and to assess right ventricular regional wall motion.

### Gated Blood Pool

In gated blood pool or gated equilibrium studies, data are collected continuously over hundreds of cardiac cycles. These data are then summed for discrete intervals of each cycle to give an average or representative picture of the patient's cardiac function. Although this approach may result in a loss of some high-frequency information that is preserved on first-pass studies, the gated technique may be more statistically accurate. Gated blood pool studies are useful for assessment of cardiac function, quantitation of ejection fraction (LVEF, RVEF), estimation of wall motion abnormalities, detection of ventricular aneurysm, detection of ventricular regurgitation, evaluation of cardiotoxicity (i.e., Adriamycin), and follow-up of medical or surgical therapy.

Gated equilibrium studies require a radiopharmaceutical that remains within the vascular space for the duration of the study. Although labeled human serum albumin (HSA) was initially used for gated equilibrium imaging, in vivo or in vitro labeling of red blood cells with technetium-99m is currently the preferred technique for the study. In vivo labeling provides a higher target-to-background ratio than is obtained with technetium-labeled albumin, which tends to slowly leach out of the vascular space and produce excessive background activity, especially in the liver.

Gated equilibrium imaging is usually performed in the anterior view to visualize the right atrium, tricuspid valve plane, right ventricle, pulmonary artery and outflow tract and the anterolateral and apical portions of the left ventricle. Imaging in the left anterior oblique (LAO) view best separates the right and left ventricles. The exact LAO angle should be optimized for each patient by selecting the angle that best separates the ventricles and provides optimal visualization of the septum. The left atrium, aortic arch, mitral valve plane and the inferior and lateral walls of both ventricles are also well visualized in this view. To avoid atrial overlap and promote optimal chamber separation, a caudal tilt may be applied to the camera. Additional views such as the left posterior oblique (LPO) and the left lateral (LL) projections may also be used if desired. These additional views allow visualization of the apex and the anterior wall and the assessment of left ventricular aneurysms.

Ensuring an adequate gating signal is an important part of this procedure since data collection depends on the physiological signal. Positioning the heart between electrodes provides an R wave signal of sufficient amplitude to trigger the camera or computer to initiate the data collection cycle. If the gating sequence is proper, all but the last frame in the cineangiogram should have the same number of counts (±10%). The use of windowing, postbeat or dynamic beat filtration will reduce or reject the acquisition of the irregular beat. To simplify matters, the study should be performed when the patient is experiencing a normal sinus rhythm.

The background contribution on a gated equilibrium image is significant, i.e., approaching 50% of the peak chamber counts. On the LAO image, the operator selects a region of interest to the right of the ventricle within the lung field that appears to be representative of the background surrounding the heart. This region should show a constant count for each frame of the study. A rise and fall in counts could indicate that the region of interest overlies a major artery which would give a less accurate background value. In this case, the operator should select another representative-background region. The background value ultimately chosen is expressed in counts per matrix element or pixel of the display. This value is then subtracted from every pixel in each image to obtain the background-corrected image.

In conventional systems, following the background correction procedure, the operator must select and ROI over the ventricle to be analyzed. The ROI may be either a rectangular box or a circle depending on the imaging system being used. Care is required in excluding the atrium, crossing over the septum into the other ventricle or including the outflow tract of the respective ventricle.

Gated equilibrium studies are normally interpreted in two phases: a subjective assessment that includes chamber sizes, configuration and regional wall motion throughout the cardiac cycle (qualitative analysis), followed by a calculated determination of ventricular function (quantitative analysis). Qualitative analysis begins by viewing the cineangiogram at a variety of cycling rates. With the heart about 5 cm in height on the display and the viewer about 1 meter from the screen, the image should be sufficiently blurred to obscure the matrix. Varying the contrast or smoothing the images enables optimal visualization of all cardiac structures.

During diastole, the right atrium is spherical in shape and contains about 60 ml of blood. If the patient has right atrial enlargement, there is a bulge in the lateral and supero-inferior dimensions of the chamber. This enlargement may reflect either right ventricular overload or tricuspid regurgitation. Atrial systole lasts only a brief time, e.g., about 100 msec at a resting heart rate.

The right ventricle in the anterior view appears triangular in shape and, at diastole, it has a volume of approximately 165 ml in a 70 kg adult. During systole, the ventricle may appear to rotate as it contracts. With an acute increase in volume load, occurring in right ventricular infarction when right heart function is impaired, the ventricle compensates by dilating. Right ventricular enlargement may reflect left-sided heart failure and pulmonary hypertension. The normal right ventricular ejection fraction is 50 to 60 percent.

The left atrium and mitral valve plane are best visualized on a 30° left posterior oblique view which is not routinely performed in most laboratories. On the usual 40° to 45° LAO view, the left atrium appears superior to the left ventricle and inferior to the left pulmonary artery. The normal chamber volume is about 40 ml. Left atrial enlargement may reflect mitral stenosis or left ventricular hypertrophy. In addition, biatrial enlargement is seen in atrial fibrillation.

The left ventricle is the hardest working chamber of the heart. It must generate about three times as much pressure as the right ventricle and its walls are approximately three times as thick as those of the right ventricle. This thickness is visualized on the blood pool image as a halo of decreased activity around the chamber. The left ventricle contains about 150 ml of blood at end-diastole. Normally 55 to 65 percent of this volume is ejected with each beat. The papillary muscles may be visible as filling defects within the chamber, which may be more apparent on the LAO view. The motion of antero- and posterolateral surfaces is usually greater than that of the septum, the apex and the inferior wall. The septum should thicken during systole and contract toward the left ventricle. Left ventricular hypertrophy is an indication of aortic stenosis or systemic hypertension.

Quantitative determinations of ventricular function are possible after correction of the data for background activity. In conventional systems, the operator defines a region of interest around the right or left ventricle. The computer generates a time-activity curve using the frames that represent the cardiac cycle. The computer will designate those frames which best represent end-diastole and end-systole. Then, the ejection fraction is calculated.

The right ventricular ejection fraction may also be calculated from the gated equilibrium data. This calculation requires particular attention to the position of the right atrium. In the 45° LAO view, the atrium may lie behind the ventricle, particularly when the patient is in the supine position. Failure to account for superimposed right atrial activity may yield an erroneously depressed ejection fraction. Maneuvers to correct for right atrial activity include tilting the camera head somewhat caudad for better chamber separation, and exercising care in selecting an accurate ROI.

To assist in assessing regional wall motion abnormalities, phase analysis can be applied which represents the timing of a ventricular contraction on a pixel-by-pixel display. During phase analysis, functional images are generated which demonstrate global or regional changes within the ventricles. Some of these images include:

The stroke image can be generated by subtracting the end-systolic image from the end-diastolic image. This stroke volume image is used to assess regional volume changes, regional ventricular function and wall motion.

The ejection fraction image is obtained by dividing the stroke volume image by the end-diastolic image. This image allows the assessment of regional EF values.

The paradox image is the inverse of the stroke volume image in which the end-diastole image is subtracted from the end-systolic image. This image is used to evaluate dyskinetic segments which reflect ventricular aneurysms.

The phase image is generated by following each pixel through the contraction periods of the atria and the ventricles. Normal images demonstrate a uniform contraction pattern while abnormal images show an area that is contracting out-of-phase from its surrounding areas.

These functional images in addition to the cine display of the gated images provide a qualitative assessment of ventricular function.

Many nuclear medicine and nuclear cardiology laboratories perform exercise blood pool studies using either the first-pass technique or the gated equilibrium procedure. These studies are useful in disclosing abnormalities in cardiac function that become apparent only with physiologic exercise. Such tests may help detect coronary disease which results as a reduced exercise ejection fraction and regional wall motion abnormalities. However, these findings may be associated with other cardiac conditions, such as cardiomyopathies and valvular disorders, resulting in poor specificity for coronary artery disease.

In conventional gated blood pool procedures, patients exercise on commercially available tables. This equipment must remain stable and motionless during the exercise portion of the study. To prevent motion artifact, the patient should be restrained on the table during exercise with his/her feet well anchored to the ergometer pedals. The ergometer should be attached to the imaging table in a way that does not interfere with imaging and which permits the patient to exercise freely. Cantilevering the ergometer off the end of the table has been found to work fairly well. In addition, the ergometer must be appropriately calibrated for graded exercise to permit workload monitoring and rpm measurement.

To minimize heart rate variations and fatigue within an imaging interval, the data collection period should be as short as possible. A 2- to 2.5-minute collection period may be used, with the patient pedaling at a rate of 50 to 70 rpm during this time. This collection period permits sufficient time within the 3-minute intervals of the exercise protocol. The heart rate tends to plateau after 60 to 90 seconds of a constant workload, which provides about 1.5 to 2 minutes of a relatively stable heart rate for data collection.

### SPECT

Single photon emission computed tomography (SPECT) is a growing diagnostic technique. SPECT imaging devices may be computer controlled for both data collection and data analysis. The detector may rotate either 180° or 360° around an axis through the center of the patient. Parallel-hole collimators are used to insure that each detector sees only the photons along a narrow band that extends from the detector to the object in the radius of rotation which is usually the patient or the organ being imaged.

Scanning of a ray across a plane in the patient at a particular angular increment, either by physical motion of the detector or by sampling of adjacent detector elements in a gamma camera, forms a projection corresponding to the count-rate profile of the subject at a specified angular orientation. After a complete set of projections has been obtained, the computer sorts the data and then reconstructs an image of the activity distribution within the plane of interest.

Since the left ventricle does not lie in any 90-degree plane to the long axis of the body, transaxial images of the left ventricle are not in a true apex-to-base orientation. The operator must reorientate transaxial slices into oblique images for both the stress and redistribution images. For example, a typical thallium study might present twenty short axis slices, fifteen vertical long axis slices and ten to fifteen horizontal long axis images for the stress and rest studies. Analysis of this abundance of information is time-consuming. However, presentation of single slices often underestimates the extent and severity of a perfusion defect over the complete volume of the left ventricle. Thus, a composite image is an alternative which displays the sum of the slices. The composite display may be produced by a technique called the bull's eye program which adds the short axis slices, from apex to base, to form a single, two-dimensional polar map of left ventricular perfusion.

As described above, first pass cardiac imaging systems involve the injection of a bolus of radiopharmaceutical into a patient and imaging the bolus on its first pass through the heart. First pass imaging allows temporal separation of the heart chambers, and permits assessment of ventricle wall motion, ES and ED ventricle volumes, LV and RV ejection fractions and pulmonary transit time. Images from a single camera location (typically an anterior location) are captured for a time period of about 60 seconds as the bolus passes through the heart. During the first pass, images are captured in the form of multiple millisecond frames for temporal resolution. First pass cardiac testing may be performed in conjunction with exercise and may be used to compare rest EF to stress EF.

A conventional first pass imaging system sold by Scinticor/Picker under the designation SIM 400 has historically been used. However, such systems suffer from several disadvantages. For example, conventional first pass systems utilize a single camera which provides only a single viewing angle of the patient's heart. It would be desirable to obtain images from multiple viewing angles simulataneously.

### SUMMARY OF THE INVENTION

An aspect of the present invention is to provide a cardiac imaging system comprising a plurality of camera heads for generating first pass and/or gated blood pool images from different viewing angles with respect to a patient.

Another aspect of the present invention is to provide a method of cardiac imaging comprising positioning a plurality of camera heads in relation to a patient and obtaining first pass and/or gated blood pool images from each of the camera heads.

These and other aspects of the present invention will be more apparent from the following description.

### BRIEF DESCRIPTION OF THE DRAWINGS

FIG. 1 illustrates a first pass and/or gated blood pool imaging system including two cameras in accordance with an embodiment of the present invention.

FIG. 2 illustrates a two-camera first pass and/or gated blood pool imaging system positioned next to a treadmill for conducting a first pass test on a patient.

### DETAILED DESCRIPTION

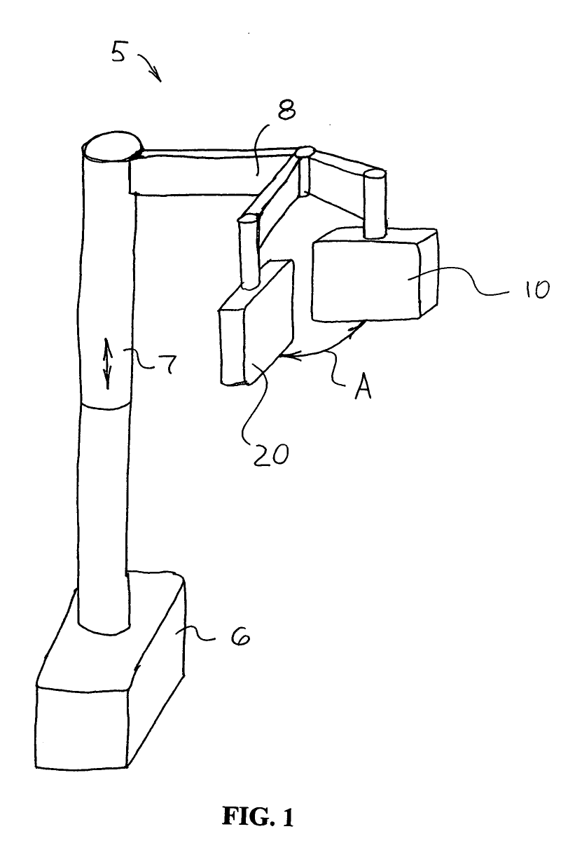

FIG. 1 schematically illustrates a two-head camera system 5 for first pass and/or gated blood pool imaging in accordance with an embodiment of the present invention. The system 5 includes a base 6, a vertical support member 7, and a swing arm 8 which supports two first pass cameras 10 and 20. The first camera 10 is typically aligned in an anterior orientation with respect to the patient. The second camera 20 may be positioned in any other desired orientation. For example, the second camera 20 may be positioned at an angle A of 90° with respect to the first camera 10, and may obtain a side image of the patient. Although two cameras are shown in FIG. 1, any other suitable number of cameras may be used. A suitable camera is commercially available from CDL Medical Tech, Inc. under the designation CDLCAM.

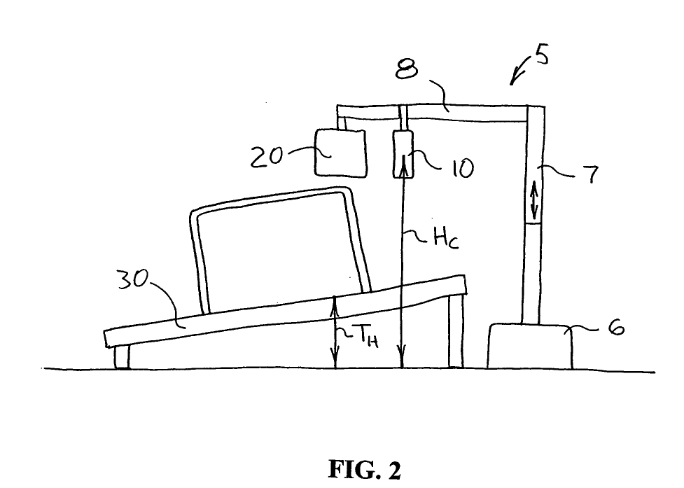

As shown in FIG. 2, the first pass and/or gated blood pool imaging system 5 may be positioned adjacent to a treadmill 30 or any other suitable exercise device. In the embodiment shown in FIG. 2, the first camera 10 is positioned in an anterior position in front of a patient (not shown) running or walking on the treadmill 30. The second camera 20 may be positioned on the side of the patient. The inclination or height T_{H} of the treadmill 30 may be adjusted during the test procedure. The height H_{C} of the cameras 10 and 20 may be adjusted to track the height T_{H} of the treadmill 30. This may be done, for example, by telescoping the vertical support member 7. In a preferred embodiment, the camera height H_{C} may be adjusted automatically to track the height T_{H} of the treadmill 30.

First pass and/or gated blood pool imaging fom multiple angles is performed as follows: Each camera simultaneously captures first pass images and/or gated blood pool images. This provides two different planes of projection, e.g., an anterior plane of projection and a left anterior oblique plane of projection, with a single first pass radioisotope injection. Physicians may thus visualize both planes of projection simultaneously to differentiate global versus regional wall motion abnormalities. The use of multiple cameras in accordance with the present invention allows images to be generated from multiple angles with a single first pass procedure. Simultaneous images are generated and multiple tests from different camera angles are eliminated.

Whereas particular embodiments of this invention have been described above for purposes of illustration, it will be evident to those skilled in the art that numerous variations of the details of the present invention may be made without departing from the invention. For example, the present multiple camera head system may be used for other diagnostic tests, such as chest pain center testing, pulmonary testing, high energy PET imaging and the like.

1. A cardiac imaging system comprising a plurality of camera heads for generating first pass and/or gated blood pool images from different viewing angles with respect to a patient.

2. The cardiac imaging system of claim 1, wherein the system comprises two of the camera heads.

3. The cardiac imaging system of claim 1, wherein each camera head generates a first pass image.

4. The cardiac imaging system of claim 1, wherein each camera head generates a gated blood pool image.

5. A method of cardiac imaging comprising:
positioning a plurality of camera heads in relation to a patient; and
obtaining first pass and/or gated blood pool images from each of the camera heads.

6. The method of claim 5, wherein two of the camera heads are positioned in relation to the patient.

7. The method of claim 5, wherein first pass images are obtained from the camera heads.

8. The method of claim 5, wherein gated blood pool images are obtained from the camera heads.
